(19) United States
(12) Patent Application Publication
(10) Pub. No.: US 2005/0031544 A1
(43) Pub. Date: Feb. 10, 2005

(54) Receptor mediated nanoscale copolymer assemblies for diagnostic imaging and therapeutic management of hyperlipidemia and infectious diseases

(76) Inventor: Philip Chidi Njemanze, Owerri (NG)

Correspondence Address: PHILIP CHIDI NJEMANZE, NO 1 URATTA/MCC ROAD, P O BOX 302, OWERI, POB302, NG

(21) Appl. No.: 10/635,820

(22) Filed: Aug. 7, 2003

(51) Int. Cl. A61K 49/00

(52) U.S. Cl. 424/9.322

(57) ABSTRACT

This invention relates to a method and system for improving diagnostic imaging and/or delivering therapeutically active agents for control of hyperlipidemia and infectious diseases (bacterial or viral), comprising nanoscale block copolymer assemblies carrying drug molecules in its core and receptor peptide in the corona surrounding the core, forming larger micelle or vesicle aggregates with target molecules such as LDL molecules and surface lipid of microorganisms.

### CROSS REFERENCE TO RELATED APPLICATION

US Patent Documents

U.S. Pat. No. 6,576,221 Jun. 10, 2003 Kresse, et al. 424/9
U.S. Pat. No. 6,548,048 Apr. 15, 2003 Cuthbertson, et al. 424/9
U.S. Pat. No. 6,555,654 Apr. 29, 2003 Todd, et al. 530/350

### STATEMENT REGARDING FEDERALLY SPONSORED RESEARCH OR DEVELOPMENT

Not applicable.

### REFERENCE TO A MICROFICHE APPENDIX

Not applicable

### BACKGROUND OF THE INVTION

This invention relates to a method and system for improving diagnostic imaging and/or delivering therapeutically active agents for control of hyperlipidemia and infectious diseases (bacterial or viral), comprising nanoscale block copolymer assemblies carrying drug molecules in its core and receptor peptide in the corona surrounding the core, forming larger micelle or vesicle aggregates with target molecules such as LDL molecules and surface lipid of microorganisms.

From archaebacteria to humans, cell membrane are self-assemblies of lipids as well as integrated and peripheral membrane proteins (biopolymers). Block copolymers have the same basic architecture as lipids but consist of distinct polymer chains covalently linked in a series of two or more segments, see description by Bates F S, Fredrickson G H, in Physics Today 1999, volume 52, page 32 and by Foster, Zisenis M, Wenz E, Antonietti M, in Journal of Chemical Physics-, 1996, volume 104, page 9956. In the presence of solvents block copolymer nanostructures swell, rearrange, and transform as lyotropic phases, see Föster et al., in Macromolecules 2001, volume 34, page 4610, and Hajduk D A, Kossuth M B, Hillmyer M A, Bates F S, in Journal of Physical Chemistry B1998, volume 102, page 4269. Vesicles are closely related to rod-like and spherical micelle morphologies in that all are solvent dependent, self-directed assemblies. Vesicles are formed from diblocks in which the hydrophobic segment not only has high glass transition temperature (e.g., polystyrene, PS), and the hydrophilic segment is ionic (e.g., polyacrylic acid, PAA), but the overall copolymer molecular weight is considerable higher than that of lipids. Using fully synthetic diblock copolymers of nonionic polyethyleneoxide-polybutadiene (PEO-PBD) and hydrogenated homolog PEO-polyethylethylene (PEO-PEE), more monomorphic, unilamellar vesicles referred to as “polymersomes” have been made under a variety of aqueous conditions see article by Discher et al, in Science 1999, volume 284, page 1143 and Lee J C-M, in Biotechnological Bioengineering 2001, volume 43, page 135. There are also triblock copolymers which are polymersome formers. A commercial triblock known as a “pluronic” with a relatively large poly(propyleneoxide) midblock (PEO5-PPO68-PEO5) yields small vesicles in water with relatively thin membranes of d=3 to 5 nm and stability of only hours, see article by Schillen K, Bryskhe K, Mel'nikova YS, Macromolecules 1999, volume 32, page 6885. Another vesicle-forming triblock consists of a hydrophobic midblock of poly(dimethylsiloxane) (PDMS) and two water-soluble blocks of poly(2-methyloxazoline) (PMOXA) terminating in a cross-linkable methacrylate groups, see article by Nardin C, Hirt T, Leukel J, Meier W, Langmuir 2000, volume 16, page 1035. Membrane proteins 3 to 5 nm high have been compatibly inserted into PDMS-PMOXA membranes see article by Meier W, Nardin C, Winterhalter M, Angew. Chem. Int. Ed. 2000, volume 39, page 4599. Inserted channel proteins can also effectively dock with viruses and facilitate transfer-loading of viral DNA into polymer vesicle, see article by Graff A, Sauer M, Gelder P V, Meier W, Proceedings of the National Academy of Sciences of the United States of America 2002, volume 99, page 5064. In one embodiment of the present invention the inserted proteins provide docking sites for viruses in blood during viremia and a means to clear the viruses from blood stream. According to the teachings of another embodiment of the present invention the inserted proteins provide docking sites to transfer genetically engineered viral DNA through the membranes of vesicles. The latter subsequently ferries the DNA to specific tissues in the body entering the cell via transduction to deliver the genetic materials for treatment of certain conditions including infectious and noninfection diseases requiring gene therapy.

Micelles for lipid-size surfactants and larger polymer superamphiphiles has been described in a book by Israelachvili J, in Intermolecular an Surface Forces published by Academic Press, in New York, 1992, second edition, and by Zhang L, Eisenberg A, in science 1995, volume 268, page 727. Micelles differ from vesicles in that they lack the shell character and encapsulated bulk solution phase of a vesicle. Phospholipid membranes when mixed with sufficient concentrations of polyethylene glycol (PEG)-modified lipids tend to generate the highly curved micelles see full description by Bedu-Addo F K, Tang P, Xu Y, Huang L, in Pharmacological Research, 1996, volume 13, page 710. When liposomes formed with PEG-lipid are injected into the blood stream they have been found to clear more slowly from the blood circulation see book by Lasic D D, Papahadjopoulos D. (Editors) entitled Medical Applications of Liposomes published by Elsevier Science, in Amsterdam, 1998. In blood, transition between aggregate morphologies as well as aggregate fluidity and stability are governed by chain molecular weight, interfacial surface tensions, and/or selective fraction of the amphiphile, see Hanley K J, Lodge T P, Huang C I, Macromolecules, 2000, volume 33, page 5918. Phospholipids segregate and form vesicles in many aqueous solutions. Lipids and small amphiphiles can differ considerably in their hydrophilic head group, but they almost always contain one or two strongly hydrophilic chains composed of multiple ethylene units (—CH2-CH2-)n (with n=5 to 18 typically). One obvious problem that arises with both micelle aggregates and polymersomes of increased size and molecular weight is fluidity: The fluidity will drop with rising molecular weight as mentioned above. One way to overcome this problem is to make vesicles and micelles with amphiphiles with molecular weight less than 1 kD. As would be discussed below it is necessary to restrict the number of target receptors sites to just a couple so that fluidity could be maintained within a certain range and at the same time rendering the numerous aggregates of vesicles or micelles contrastable in vessels on high resolution images of ultrasound and magnetic resonance imaging

Nanoscale containers are formed from partitioning a hydrophobic drug within the core of a micelle formed by self-assembly of an AB block copolymer. The hydrophobic B domains self-associate into a core to escape contact with water, pushing the hydrophilic A domains into a corona surrounding the core. This yields spherical micelles 20-45 nm in diameter. Water soluble biocompatible nanocontainers comprising block copolymer micelles used for delivering hydrophobic drugs have been described by Savic R, Luo L, Eisenberg A, Maysinger D, in Science, 2003, volume 300, page 615. Cholesterol is an intriguing component of cell membranes because it both toughens and fluidizes as discussed by Lipowsky R, Sackmann E, (editors) in a book entitled Structure and Dynamics of Membranes-from cells to Vesicles, published by Elsevier Science in Amsterdam, 1995.

Fats are hydrolyzed to fatty acids, 2-monoglycerides and glycerol. These are absorbed only from the small intestine, largely in the duodenum and jejunum. The presence of bile salts is essential for the absorption of long chain fatty acids and 2-monoglycerides. The bile salts forms micelles, which are molecular aggregates containing a hydrophilic surface at the interface with aqueous phase, and a lipophilic core within which fatty acids and 2-monoglycerides and other lipoid substances such as cholesterol and the fat-soluble vitamins are accumulated. The free fatty acids have high floatation constants (Sƒ) expressed in negative Svedberg units as described by Bowman W C, and Rand M J in a book Textbook of Pharmacology, Second Edition published by Blackwell Scientific Publication, Oxford, pages 1.28 and 28.32.

Elevated concentration of circulating free fatty acid has been reported by Jouven X, Charles M A, Desnos M et al. in Circulation 2001, volume 104, page 756, to be an independent risk factor for sudden death in middle aged men in a long term cohort study. About three fourths of the total cholesterol in normal human plasma is contained within LDL particles. LDL supplies cholesterol to a variety of extrahepatic parenchymal cells, such as adrenal cortical cells, lymphocytes and renal cells. These cells have LDL receptors localized on the cell surface. LDL that binds to this receptor is taken up by receptor mediated endocytosis and digested by lysosomes within the cell. Extrahepatic tissues and the liver have abundant LDL receptors. In humans, 70 to 80 percent of LDL is removed from plasma by the LDL receptor pathway. A number of diseases are caused by elevated concentration of triglycerides or cholesterol in fasting plasma, a condition called hyperlipidemia

It has been clear for several decades that elevated blood cholesterol is a major risk factor for coronary heart disease (CHD) and stroke. The relationship of between increased carotid intima media-thickness (IMT) and high cholesterol has been demonstrated by Allan P L, Mowbray P I, Lee A J, Fowkes F G, in Stroke, 1997, volume 28, page 348; and also by Chambless L E, Heiss G, Folsom A R, Rosamond W, Szklo M, Sharrett A R, Clegg L X, in American Journal of Epidemiology, 1997, volume 146, page 483. Similarly, high cholesterol is a major risk factor for development of symptomatic and asymptomatic peripheral arterial disease as described by Zheng Z J, Sharrett A R, Chambless L E, Rosamond W D, Nieto F J, Sheps D S, Dobs A, Evans F W, Heiss G, in Atherosclerosis, 1997, volume 131, page 115.

Many studies have shown that the risk of CHD and stroke events can be reduced by lipid lowering therapy. The first inhibitor of HMG-CoA reductase: lovastatin (MEVACOR.RTM.; see U.S. Pat. No. 4,231,938); simvastatin (ZOCOR.RTM; see U.S. Pat. No. 4,444,784), pravastatin sodium salt (PRAVACHOL.RTM.; see U.S. Pat. No. 4,346,227), fluvastatin sodium salt (LESCOL.RTM.; see U.S. Pat. No. 5,354,772), atorvastatin calcium salt (LIPITOR.RTM.; see U.S. Pat. No. 5,273,995) and cerivastatin sodium salt (also known as rivastatin; see U.S. Pat. No. 5,177,080). The structural formulas of these and additional HMG-CoA reductase inhibitors, are described by Yalpani M., published in Chemistry & Industry, Feb. 5, 1996, page 85. The HMG-CoA reductase inhibitors described above belong to a structural class of compounds which contain a moiety which can exist as either a 3-hydroxy lactone ring or as the corresponding ring opened dihydroxy open-acid, and are often referred to as “statins” Most H MG-CoA reductase drugs (simvastatin by way of example) undergoes extensive first-pass extraction in the liver, its primary site of action, with subsequent excretion of drug equivalents in bile. As a consequence of extensive hepatic extraction of simvastatin (estimated to be >60% in man), the bioavailability of drug to the general circulation is low. In one study cited in the Physicans' Desk Reference, 1999, page 1922, in a single dose study in nine healthy subjects, it was estimated that less than 5% of an oral dose of simvastatin reaches the general circulation as active inhibitors.

However, until date determination of cholesterol has been performed in blood sample analysis using standard laboratory methods as has been described by Tietz N W (editor) in a book entitled Fundamental of Clinical Chemistry published by W B Saunders in Philadelphia, Pa., 1976. Otherwise diagnostic imaging has focused on plaques which are partly a consequence of high cholesterol. Diagnostic imaging of plaques in carotid arteries has usually used invasive digital subtraction angiography (DSA). Minimally invasive computerized tomography has been used as described by Anderson G B, Ashforth R, Steinke D E, Ferdinandy R, Findlay M, in Stroke, 2000, volume 31, page 2168. The use of noninvasive ultrasound has been described by De Bray J M, Glatt B, in Cerebrovascular Diseases, 1995, volume 5, page 414. Recently the improved use of magnetic resonance angiography has been applied in clinical practice as described by Leiner T, van Engelshoven J MA in Diagnostic Imaging Europe, March/April, 2002, page 16.

However, no currently used imaging technique could be applied to image circulating lipids for diagnostic purposes. As a result of this physicians have only indication to treat patients when their blood lipoproteins rise to a certain levels. The classification of lipid profile was defined according to the guidelines of the National Cholesterol Education Program (NCEP) and the more recent Adult Treatment Panel III guidelines for cholesterol management, National Cholesterol Education Program, in Circulation 2002, volume 106, page 3143. The obvious disadvantage is that only total serum or plasma values are determined with no tissue differential distribution of cholesterol established for the critical organs of the heart, brain and kidneys. Diagnosis is often in the late stage after plaques have deposited at sites causing major occlusions.

Nanoparticles have been developed for diagnostic purposes. U.S. Pat. No. 6,576,221 to Kresse et al. describes iron-containing nanoparticles having a modular structure, their production, and their use for diagnostic and therapeutic purposes. The nanoparticles according to the '221 patent are characterized in that they consist of an iron-containing core, a primary coat (synthesis polymer), and a secondary coat (targeting polymer) and, optionally, of pharmaceutic adjuvants, pharmaceuticals, and/or adsorption mediators/enhancers. The '221 patent is incorporated herein by reference. However, prior art focuses on using iron-containing core as the contrast agent and could not be applied for the purposes outlined in the present invention. There is no direct imaging of the causative agent such as LDL-cholesterol or microorganisms and as such lacks specificity for this purpose.

The copolymer micelles could be designed to carry the drug in its core and the LDL-receptor at its corona with attached LDL molecules. The core of the micelle could in addition carry a multiplicity of drug options such as inhibitor of HMG-CoA reductase, recombinant tissue plasminogen activator (rtPA), and antagonists of N-methyl-D-aspartate (NMDA) receptors. U.S. Pat. No. 6,468,219 to Njemanze described an implanted system with possibility for multidrug delivery in an instant of detection of microembolic signals suggestive of a stroke. Such applications could be used to deliver nanoscale copolymer micelles with multidrug combinations in the event of a stroke.

Mechanisms have been developed to disrupt the endosomal membrane even more specifically. For example, peptides that induce fusion or lysis of membrane vesicles as described by Wagner E. in Advanced Drug Delivery, 1999, volume 38, page 279, could be incorporated into such nanoscale assemblies. As the reach target cells, these agents are highly soluble at pH 7.4 and form amphiphiles then destabilize as the pH in the endosome is lowered before lysosomal fusion. The amphiphiles then destabilize the endosomal membrane, allowing permeation of the incorporated drug. The incorporated peptides in the corona for fusion and lysis could be made to be tissue specific. For example, to facilitate use of inhibitors HMG-CoA reductase for treatment of breast cancer or prostate cancer without disrupting whole body fat homeostasis, the fusion peptides are selected that are specific for breast and prostatic tissues respectively. This will assure fusion to target cells and subsequent drug delivery.

The micelle mediated mechanisms described above involve endocytosis and subsequent endosomal permeation or destabilization. It may also be possible to use biological mechanisms to enter the cell through a pathway other than endocytosis. One such mechanism involves transduction as has been described by Schwarze S R, Hruska K A, Dowdy S F, in Trends in Cell Biology 2000, volume 10, page 290. Some proteins, including the HIV TAT protein, contain “protein transduction domains,” which cause the parent protein to cross the membrane directly. Attachment of such peptide to oligonucleotides and proteins induces their direct transport across the membrane in a manner that is not particularly sensitive to the molecular identity of the cargo. For example, when synthetic, drug conjugated polymers were decorated with the transduction domain peptide from the TAT protein, the polymers were directly transduced across the plasma membrane as described by Jensen K D, Nori A, Tijerina M, Kopeckova P. Kopecek J in Journal of Control Release 2003, volume 87, page 89, without endocytosis or passage through the lysosome, carrying drug directly into the cytoplasm. Furthermore, when surface-cross-linked micelles were similarly grafted with TAT peptide, the micelle seemed to be transduced across the membrane as well as described by Liu J Q, Zhang Q, Remsen E F, Wooley K L, in Biomacromolecules 2001%, volume 2, page 362.

The drugs may also be enclosed in vesicles rather than micelles. Vesicles are microscopic sacs that enclose a volume with a molecular thin membrane. The membrane are generally sell directed assemblies of amphiphilic molecules with dual hydrophilic-hydrophobic character. Biological amphiphiles form vesicles central to cell function and are principally lipids of molecular weight less than 1 kilo dalton. Block copolymers that mimic lipid amphiphilicity can also self assemble into vesicles in dilute solution, but polymer molecular weights can be orders to magnitude greater than those of lipids. Vesicles which enclose an aqueous core rather than micelles may permit larger molecules to be incorporated in a general way, independent of the identity of the molecule as discussed by Discher D E, Eisenberg A, in Science 2002, volume 297, page 967. According to the teachings of the present invention the resulting polymer vesicle with drug incorporated in aqueous core could be visualized on magnetic resonance imaging as hyperintense signals or echogenic signals on ultrasound images.

One function of LDL is to supply cholesterol to tissues such as the adrenal cortical cells, lymphocytes, and renal cells. These cells have LDL receptors localized on the cell surface. LDL that binds to this receptor is taken up by receptor endocytosis and digested by lysosomes within the cells. The cholesteryl esters of LDL are hydrolyzed by a lysosomal cholesteryl esterase (acid lipase), and the liberated cholesterol is used for membrane synthesis, as a precursor for steroid hormone synthesis, and as a regulatory molecule that suppresses the synthesis of new LDL receptors. The present invention uses specific fusion or lysis proteins to target the tissues where reduction of LDL molecules is desirable. In other words, the fusion and lysis proteins are tissue specific. According to the teachings of the present invention some micelle assemblies which are intended to deliver LDL molecules to specific tissues are designed not to carry a drug in its core, instead carry a placebo substance. A placebo is a substance that has no therapeutic effect for the known disease process. In other words there tissue differential elimination of cholesterol. The tissue specific targeting using fusion proteins and drug delivery could prevent serious side effects, for example myalgia caused by statins. The pharmacological composition of the drug included in the core is chosen by indication for the disease process but could also include drug combinations used for closely associated complications. For example, diabetes could be associated with hyperlipidemia and drugs indicated for both conditions could be packaged together in some formulations.

The circulating fatty globulets are slow moving nanospheres and are displaced to the outer curvature of the aortic arch, carotid bulb and at vascular bifurcations by centrifugal forces. A double vortex circulation is superimposed on the main flow pattern leading to helical type motion similar to observations made in vivo using cinephotographic analysis of aortic and major arterial flow patterns by Rogers W H, Rukskul A, Camishion R C, Padula R T, published in Archives of Surgery 1971, volume 103, page 93. The centrifugal forces that develop at the curvature of the ascending aorta ‘sucks’ the nanospheres into the first available outer opening which is the brachiocephalic artery and then into the right common carotid artery, where they rise and are stopped by the carotid bifurcation flow divider. Accumulation of plaque at the carotid bulb is influenced by ‘flow separation’ and other flow phenomena as described by Fung Y C in a book Biodynamics Circulation, published by Springer-Verlag, New York, 1984, page 153.

Most strokes occur first in small vessels and therefore are called small vessel diseases, however, lack of proper imaging techniques have hampered efforts to document the process of thrombosis in small vessels caused by circulating LDL molecules and their clinical evolution in stroke. The present invention provides a method that will identify circulating LDL molecules in small vessels in the brain by providing highly contrasted magnetic resonance images. This will complement images presently obtained using diffusion weighted magnetic resonance images in stroke diagnosis.

Until now there is no clinical applicable method for imaging circulating free fatty acids. U.S. Pat. No. 6,548,048 to Cuthbertson, et al. describes a diagnostic and/or therapeutically active agent comprising gas microbubbles, more particularly to such agents comprising lipopeptide stabilized gas microbubbles. One preferred aspect of the '048 patent, there is targeting of ultrasound microbubbles for disease imaging and drug delivery. Thus, viewed from another aspect the '048 patent provides a targeted diagnostic and/or therapeutically active agent, e.g. an ultrasound contrast agent, comprising (i) gas filled microbubbles stabilized by membrane forming amphiphilic lipopeptides capable of interacting with ultrasound irradiation to generate a detectable signal; (ii) one or more vector or drug molecules or; combination of both, where said vector(s) have affinity for a particular target site and/or structures within the body, e.g. for specific cells or areas of pathology; and (iii) one or more linkers connecting said microbubbles and vectors, in the event that these are not directly joined. According to the teachings of the '048 patent, the microbubbles may be coupled to vectors such as monoclonal antibodies which recognize specific target areas or to a secondary antibody which has a specificity for a primary antibody which in turn has specificity for a target area. Further more the '048 patent aswell as WO-A-9818501 use microbubbles in drug delivery applications. The '048 patent is incorporated herein by reference. A comprehensive summary of known vectors and linking groups useful in targeting ultrasonic echography can be found in International Patent Publication No. WO-A-9818501. Prior art uses microbubbles which provides the contrast medium for ultrasound. while in terms of specificity of the vector prior art provides targets however, the image is determined by the present of the microbubble which lacks specificity when visualized in the blood stream according to the teachings of patent '048. The present invention on the other hand, provides a tool for therapeutic drug delivery in combination with specific receptor-mediated contrast enhancement suitable for ultrasound and magnetic resonance imaging. The contrast enhancement is based on specific interaction between the aggregates and the ultrasound irradiation or magnetic resonance to generate a detectable signal. The present invention does not mandate use of microbubbles or metal ions as contrast enhancing agents. The present invention uses block copolymer micelles as biocompatible nanocontainers for delivering therapeutic drug. By “therapeutic drug” is meant an agent having a beneficial effect on a specific disease in a living human or non-human animal. The living human or non-human is hereafter referred to as a ‘patient’. Combinations of drugs and ultrasound contrast agents have been proposed in, for example, WO-A-9428873 and WO-A-9507072, these products lack vectors having affinity for particular sites and thereby show comparatively poor specific retention at desired sites prior to or during drug release. Similarly, the '048 patent describes a vector mediated direction but is impractical to distinguish image enhancement due to gas microbubbles by themselves and those due to specific vector mediated direction in the blood stream. Therefore the '048 patent lacks the necessary specificity to characterize the images for application in LDL determination in blood. Since the present invention does not use gas microbubbles or iron containing core only circulating LDL receptor-mediated binding to LDL molecules provides enhancement. The image seen is highly specific and reflects qualitative and quantitative characterization of the LDL complexes circulating in blood. Quantitative assessment of the number of circulating micelle aggregates with LDL molecules and also vesicles could be performed with microembolic signal detection algorithm using transcrania Doppler ultrasound according to the concensus on microembolus detection criteria described by Ringelstein E B, Droste D W, Babikian V L, Evans D H, Grosset D G, Kaps M, Markus H S, Russell D, Siebler M, in Stroke 1998, volume 29, page 725.

The micelles aggregates formed by interaction of LDL-receptor in the corona and the circulating LDL molecules comprise contrast agent as they are transported in blood through the aorta, heart and renal vessels. The contrasted ultrasound images of flowing LDL-micelle complexes in the aorta could be seen using transthoracic and transesophageal echocardiography and magnetic resonance imaging. The contrast settings and imaging adjustments of the ultrasound equipment could be made to facilitate proper visualization. Similarly, magnetic resonance images depending on the choice of repetition times, echo times and flip angles would display hyperintense signals within the arterial lumen due to contrast from LDL-micelle complexes and complexes from vesicles. Hemodynamic phenomena such as boundary condition flow, secondary flows, helical flow vortices that may create distortions and impose high shear stress along the medial walls leading to the formation of atheromata as described by Fry D L in an article in a book by P. Scheinberg (editor), entitled Cerebrovascular Diseases, published by Raven Press in New York, 1976, page 77, could be visualized with flow sensitive algorithms on magnetic resonance angiography.

The teachings of the present invention could be applied in a number of areas. One of such modifications relate to the use of the same method and system according to the teachings of the present invention for diagnosis and treatment of bacterial and viral infections. Bacteria and viruses have either lipopolysaccharide or glycoprotein complexes in the coat or envelope respectively. These protein and polysaccharide complexes are species specific and have known surface receptors. For example, the gp120 outer membrane for HIV-1. It is feasible to use block copolymers micelles to deliver antibacterial and antiviral drugs and incorporate lipopolysaccharide or glycoprotein receptors in the corona such that the bacteria and virus dock to their respective surface receptors and are ferried in blood by micelle aggregates. The microorganism-micelle complexes in significant number on the micelle aggregates are echogen because of high fat content of the lipopolysacchride or glycoprotein outer membrane and could also be seen as high intensity signals on magnetic resonance images. Thereby rendering bacteria and viruses visible in circulating blood. This permits imaging of conditions such bactermia and viremia Antibacterials and antiviral agents could also be delivered in a tissue specific manner using a fusion and lysis proteins with affinity to target sites such as endothelial organs including lymph nodes and spleen where the immune system and the drugs would act to kill the microorganisms. This may also target antibacterial and antiviral agents to specific tissues such as skin for skin infection and lungs for pneumonia using tissue specific fusion and lysis proteins for delivery to these sites while ignoring other tissues unaffected by the pathological process. The drug inclusions in the core or corona of the micelle could include antibiotics or their combinations. Similarly, antiviral medications including interferon could be incorporated into the core or corona of the micelle for tissue specific delivery.

A number of pharmacological compositions are then feasible within the core of the micelle depending on indications. The active ingredient could be selected from the group consisting of analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta.-Blockers, cardiac inotropic agents, corticosteroids, diuretics, anti-parkinsonian agents, gastrointestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof. This targeted selections may prevent drug side effects and toxicity due to interactions in multidrug regimen. It is also feasible to prevent single and multi-drug resistance since the microorganisms are targeted into such a way that lethal high dose are reached in a limited tissue site with no time for mutations into drug resistant strains.

The packaging of these drugs in the core of the micelle does not prevent formulation of their composition for immediate release, pulsatile release, controlled release, extended release delayed release, target release or targeted delayed release.

The suitable hydrophobic active ingredients are selected from a group consisting of analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Specific, non-limiting examples of suitable hydrophobic micelle core active ingredients are: acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, furazolidone, gabapentin, gemfibrozil, glibenclamide, glipizide, glyburide, glimepiride, griseofulvin, halofantrine ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. Of course, salts, isomers and derivatives of the above-listed hydrophobic active ingredients may also be used, as well as mixtures.

Among the above-listed hydrophobic active ingredients, preferred active ingredients include: acetretin, albendazole, albuterol, aminoglutethimide, amiodarone, amlodipine, amphetamine, amphotericin B, atorvastatin, atovaquone, azithromycin, baclofen, benzonatate, bicalutanide, busulfan, butenafine, calcifediol, calcipotriene, calcitriol, camptothecin, capsaicin, carbamezepine, carotenes, celecoxib, cerivastatin, chlorpheniramine, cholecaliferol, cimetidine, cinnarizinc, ciprofloxacin, cisapride, citrizine, clarithromycin, clemastine, clomiphene, codeine, coenzyme Q10, cyclosporin, danazol, dantrolene, dexchlorpheniramine, diclofenac, digoxin, dehydroepiandrosterone, dihydroergotamine, dibydrotachysterol, dirithromycin, donepezil, efavirenz, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, furazolidone, gabapentin, gemfibrozil, glibenclamide, glipizide, glyburide, glimepiride, griseofulvin, halofantrine, ibuprofen, irinotecan, isotretinoin, itraconazole, ivermectin, ketoconazole, ketorolac, lamotriginc, lansoprazole, leflunomide, loperamide, loratadine, lovastatin, L-thryroxine, lutein, lycopene, mifepristone, mefloquine, megestrol acetate, methdone, methoxsalen, metronidazole, miconazole, midazolam, miglitol, mitoxantrone, medroxyprogesterone, montelukast, nabumetone, nalbuphine, naratriptan, nelfinavir, nilutanide, nitrofurantoin, nizatidine, omeprazole, oestradiol, oxaprozin, paclitaxel, paracalcitol, pentazocine, pioglitazone, pizofetin, pravastatin, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sibutramine, sildenafil citrate, simvastatin, sirolimus, spironolactone, sumatriptan, tacrine, tacrolimus, tamoxifen, tamsulosin, targretin, tazarotene, teniposide, terbinafine, tetrahydrocannabinol, tiagabine, tizanidine, topiramate, topotecan, toremifene, tramadol, tretinoin, troglitazone, trovafloxacin, verteporfin, vigabatrin, vitamin A, vitamin D, vitamin E, vitamin K, zafirlukast, zilcuton, zolmitriptan, zolpidem, zopiclone, pharmaceutically acceptable salts, isomers and derivatives thereof, and mixtures thereof. Particularly preferred hydrophobic active ingredients include: acetretin, albuterol, aminoglutethimide, amiodarone, amlodipine, amprenavir, atorvastatin, atovaquone, baclofen, benzonatate, bicalutanide, busulfan, calcifediol, calcipotriene, calcitriol camptothecin, capsaicin, carbamezepine, carotenes, celecoxib, chlorpheniramime, cholecaliferol, cimetidine, cinnarizine, cisapride, cetirizine, clemastine, coenzyme Q10, cyclosporin, danazol, dantrolene, dexchlorpheniramine, diclofenac, dehydroepiandrosterone, dihydroergotamine, dihydrotachysterol, efavirenz, ergocalciferol, ergotamine, essential fatty acid sources, etodolac, etoposide, famotidine, fenofibrate, fexofenadine, finasteride, fluconazole, flurbiprofen, fosphenyloin, frovatriptan, furazolidone, glibenclamide, glipizide, glyburide, glimepiride, ibuprofen, irinotecan, isotretinoin, itraconazole, ivermectin, ketoconazole, ketorolac, lamotrigine, lansoprazole, leflunomide, loperamide, loratadine, lovastatin, L-thryroxine, lutein, lycopene, medroxyprogesterone, mifepristone, megestrol acetate, methoxsalen, metronidazole, miconazole, miglitol, mitoxantrone, montelukast, nabumetone, naratriptan, nelfinavir, nilutanide, nitrofurantoin, nizatidine, omeprazole, oestradiol, oxaprozin, paclitaxel, paracalcitol, pioglitazone, pizofetin, pranlukast, probucol, progesterone, pseudoephedrine, rabeprazole, raloxifene, rofecoxib, repaglinide, rifabutine, rifapentine, rimexolone, ritanovir, rizatriptan, rosiglitazone, saquinavir, sildenafil citrate, simvastatin, sirolimus, tacrolimus, tamoxifen, tamsulosin, targretin, tazarotene, teniposide, terbenafine, tetrahydrocannabinol, tiagabine, tizanidine, topiramate, topotecan, toremifene, tramadol, tretinoin, troglitazone, trovafloxacin, ubidecarenone, vigabatrin, vitamin A, vitamin D, vitamin E, vitamin K, zafirlukast, zileuton, zolmitriptan, pharmaceutically acceptable salts, isomers and derivative thereof, and rmixtures thereof. Most preferred hydrophobic active ingredients include: amlodipine, amprenavir, atorvastatin, atovaquone, celecoxib, cisapride, coenzyme Q10, cyclosporin, famotidine, fenofibrate, fexofenadine, finasteride, ibuprofen, itraconazole, lansoprazole, loratadine, lovastatin, megestrol acetate, montelukast, nabunetone, nizatidine, omeprazole, oxaprozin, paclitaxel, paracalcitol, pioglitazone, pranlukast, progesterone, pseudoephedrine, rabeprazole, rapamycin, rofecoxib, repaglinide, rimexolone, ritanovir, rosiglitazone, saquinavir, sildenafil citrate, simvastatin, sirotimus, tacrolimus, tamsulosin, teniposide, terbenafine, tetrahydrocannabinol, tiagabine, tizanidine, tramadol, troglitazone, vitamin A, vitamin D, vitamin E, zafiriukast, zileuton, pharmaceutically acceptable salts, isomers and derivatives thereof, and mixtures thereof.

In the corona of the nanoscale containers according to the teachings of the present invention, a receptor such as the LDL receptor is incorporated. U.S. Pat. No. 6,555,654 to Todd et at. describes a novel receptor, “LDL-receptor related protein-3” (“LRP-3”), along with encoding nucleic acid. The gene is associated with type 1 diabetes (insulin dependent diabetes mellitus), and experimental evidence provides indication that it is the IDDM susceptibility gene IDDM4. The '654 patent provides nucleic acid, including coding sequences, oligonucleotide primers and probes, polypeptides, pharmaceutical compositions, methods of diagnosis or prognosis, and other methods relating to and based on the gene, including methods of treatment of diseases in which the gene may be implicated including elevation of free fatty acids or hypercholesterolemia. The '654 patent is incorporated herein by reference. According to the teachings of the present invention the LRP-3 could be used as a receptor within the corona of the nanoscale container. It is used to attach the circulation LDL molecules to the nanoscale micelle so micelle aggregates could be formed. Prior art has no implemented these receptors for such applications.

The corona may also carry hydrophilic drugs as well as receptors. This brings the target molecule in close contact with the drug in the hydrophilic partition. Likewise, the hydrophilic active ingredient can be a cytokine, a peptidomimetic, a peptide, a protein, a toxoid, a serum, an antibody, a vaccine, a nucleoside, a nucleotide, a portion of genetic material, a nucleic acid or a mixture thereof.

Specific, non-limiting examples of suitable hydrophilic active ingredients that could potentially be enclosed in the corona of the micelle are selected from: acarbose, acyclovir, acetyl cysteine, acetylcholine chloride, alatrofloxacin, alendronate, aglucerase, amantadine hydrochloride, ambenomium; amifostine, amiloride hydrochloride, aminocaproic acid, amphotericin B, antihemophilic factor (human), antihemophilic factor (porcine), antihemophilic factor (recombinant), aprotinin, asparaginase, atenolol, atracurium besylate, atropine, azithromycin, aztreonam, BCG vaccine, bacitracin, becalermin, belladona, bepridil hydrochloride, bleomnycin sulfate, calcitonin human, calcitonin salmon, carboplatin, capecitabine, capreomycin sulfate, cefamandole nafate, cefazolin sodium, cefepime hydrochloride, cefixime, cefonicid sodium, cefoperazone, cefotetan disodium, cefotaxime, cefoxitin sodium, ceftizoxime, ceftriaxone, cefuroxime axetil, cephalexin, cephapirin sodiuir cholera vaccine, chorionic gonadotropin, cidofovir, cisplatin, cladribine, clidinium bromide clindamycin and clindamycin derivatives, ciprofloxacin, clodronate, colistimethate sodium, colistin sulfate, corticotropin, cosyntropin, cromolyn sodium, cytarabine, dalteparin sodium, danaparoid, desferrioxamine, denileukin diflitox, desmopressin, diatrizoate meglumine and diatrizoate sodium, dicyclomine, didanosine, dirithromycin, dopamine hydrochloride, dornase alpha, doxacurium chloride, doxorubicin, etidronate disodium, enalaprilat, enkephalin, enoxaparin, enoxaprin sodium, ephedrine, epinephrine, epoetin alpha, erythromycin, esmolol hydrochloride, factor IX, famciclovir, fludarabine, fluoxetine, foscarnet sodium, ganciclovir granulocyte colony stimulating factor, granulocyte-macrophage stimulating factor, growth hormones—recombinant human, growth hormone—bovine, gentamycin, glucagon, glycopyrolate, gonadotropin releasing hormone GnRH and synthetic analogs thereof, gonadoretin, grepafloxacin, hemophilus B conjugate vaccine, Hepatitis A virus vaccine inactivated, Hepatitis B virus vaccine inactivated, heparin sodiun, indinavir sulfate, influenza virus vaccine, interleukin-2, interleukin-3, insulin-human, insulin lispro, insulin procine, insulin NPH, insulin aspart, insulin glargine, insulin detemir, interferon alpha, interferon beta, ipratropium bromide, ifosfamide, Japanese encephalitis virus vaccine, lamivudine, leucovorin calcium, leuprolide acetate, levofloxacin, lincomycin and lincomycin derivatives, lobucavir lomefloxacin, loracarbef, mannitol, measles virus vaccine, meningococcal vaccine, menotropins, mepenzolate bromide, mesalamine, methenamine, methotrexate, methscopolamine, metformin hydrochloride, metoprolol, mezocillin sodium, mivacurium chloride, mumps viral vaccine, nedocromil sodium, neostigmine bromide, neostigmine methy sulfate, neurontin, norfloxacin, octreotide acetate, ofloxacin, olpadronate, oxytocin, pamidronate disodium, pancuronium bromide, paroxetine, perfloxacin, pentamidine isethionate, pentostatin, pentoxifylline, periciclovir, pentagastrin, pentholamine mesylate, phenylalanine, physostigmine salicylate, plague vaccine, piperacillin sodium, platelet derived growth factor-human, pneumococcal vaccine polyvalent, poliovirus vaccine inactivated, poliovirus vaccine live (OPV), polymyxin B sulfate, pralidoxime chloride, pramlintide, pregabalin, propafenone, propenthaline bromide, pyridostigmine bromide, rabies vaccine, residronate, ribavarin, rimantadine hydrochloride, rotavirus vaccine, salmeterol xinafoate, sinealide, small pox vaccine, sotalol, somatostatin, sparfloxacin, spectinomycin, stavudine, streptokinase, streptozocin, suxamethonium chloride, tacrine hydrochloride, terbutaline sulfate, thiopeta, ticarcillin, tiludronate, timolol, tissue type plasminogen activator, TNFR:Fc, TNK-tPA, trandolapril, trimetrexate gluconate, trospectinomycin, trovafloxacin, tubocurarine chloride, tumor necrosis factor, typhoid vaccine live, urea, urokinase, vancomycin, valacyclovir, valsartan, varicella virus vaccine live, vasopressin and vasopressin derivatives, vecuronium bromide, vinblastine, vincristine, vinorelbine, vitamin B12, warfarin sodium, yellow fever vaccine, zalcitabine, zanamivir, zolendronate, zidovudine, pharmaceutically acceptable salts, isomers and derivatives thereof, and mixtures thereof.

### SUMMARY OF THE INVENTION

This invention relates to a method and system for improving diagnostic imaging and/or delivering therapeutically active agents such as for control of hyperlipidemia and infectious diseases, comprising nanoscale micelle carrying drug molecules in its core and receptor peptide in the corona surrounding the core, forming larger micelle aggregates with target molecules such as LDL molecules and surface lipids of microorganisms.

The special embodiment of this invention is illustrated in the specification, it includes block and schematic diagrams for the format of the invention, and how the use the invention is shown by way of example. The system comprises a block copolymer micelles as biocompatible nanocontainers for delivering drug (by way of example, inhibitor of HMG-CoA reductase) contained at its core and attaching LDL receptor such as LRP-3 at its corona, which in turn binds circulating LDL molecules in blood (under conditions of fasting or as tolerance tests with high fat diet), an imaging system (Color flow Doppler, B-mode and/or transcranial Doppler ultrasound system, magnetic resonance imaging), with appropriate image processing software.

The present invention uses already described nanoscale polymer assemblies developed for drug delivery. The LDL receptor such as LRP-3 by way of example is incorporated in its corona. The latter allows for binding of LDL molecules in blood, forming even larger micell aggregates. These micelle aggregates comprise lipoprotein based contrast agent suitable for imaging with ultrasound and magnetic resonance. As they reach target cells, these agents are highly soluble at pH 7.4 and form amphiphiles. The amphiphiles then destabilize the endosomal membrane, allowing permeation of the incorporated drug. The present invention uses specific fusion or lysis proteins to target the tissues where reduction of LDL molecules is desirable. In other words, the fusion and lysis proteins are tissue specific. Similar application could be used for concentration of drugs in tissues affected by cancer. Specific receptors located in breast, ovarian or prostate cancer by way of example, makes it possible to apply the teachings of the present invention for treatment of these lesions.

Similarly, the present invention uses block copolymer vesicles. The therapeutically active agent may also be enclosed in vesicles rather than micelles. Vesicles are microscopic sacs that enclose a volume with a molecular thin membrane. The membrane are generally self directed assemblies of amphiphilic molecules with dual hydrophilic-hydrophobic character. Membrane proteins 3 to 5 nm high have been compatibly inserted into membranes of vesicles. Inserted channel proteins can also effectively dock with viruses and facilitate transfer loading of viral DNA into polymer vesicle. In one embodiment of the present invention the inserted proteins provide docking sites for viruses in blood during viremia and a means to clear the viruses from blood stream. According to the teachings of another embodiment of the present invention the inserted proteins provide docking sites to transfer genetically engineered viral DNA through the membranes of vesicles before introduction into the blood stream if the patient. The latter subsequently ferries the DNA material to specific tissues in the body entering the cell via transduction to deliver the genetic material for treatment of certain conditions including infectious and noninfectious diseases requiring gene therapy. The latter approach precludes the use of the whole virus and introduction into the blood stream and prevents serious complications of gene therapy.

One object of the present invention is to use block copolymer micelles as biocompatible nanocontainers for delivering drug (by way of example, inhibitor of HMG-CoA reductase) contained at its core and attaching LDL receptors at its corona.

A further object of the present invention to use copolymer micelles carrying the drug in its core and the receptor at its corona to deliver in higher concentration of the drug to the general circulation to prevent excessive extraction by the liver. Thereby improving the bioavailability of the drug.

A further object of the present invention to use block copolymer vesicles carrying an aqueous drug in its core and the receptor at its membrane to deliver in higher concentration of the drug. Thereby improving the bioavailability of the drug and preventing drug resistance.

Another object of the present invention is to use the LDL receptor to bind circulating LDL molecules in blood. The resulting copolymer micelle aggregates are echogenic due to high fat content on ultrasound and generate hyperintense signals on magneic resonance imaging within arteries.

A further object of the present invention is its use to study invivo pharmacokinetics of drugs. The copolymer micelles carrying the drug in its core and the LDL-receptor at its corona with attached LDL molecules will be transported through an organ such as the liver, and by comparing images taken of the hepatic vessels before and after administration, it plausible to estimate the amount of drug reaching the organ sites.

A further object of the present invention is its application for qualitative and quantitative estimation of circulating free fatty acids.

Another object of the present invention is to provide contrast images of free circulating LDL molecules which can be obtained under fasting conditions and at specific times after consumption of a specified amount of fatty foods and hence a quantitative analysis of dyslipidemia.

A further object of the present invention is its application for the study of hemodynamics and mechanisms of plaque formation in the carotid artery, the heart, aorta and renal vessels. According to the teachings of the present invention, flow phenomenon as a result of the circulating micelle aggregates could be observed using color flow Doppler and F mode ultrasound.

A further object of the present invention is its application for the visualizing self contrasting circulating LDL molecules after a stroke.

A further object of the present invention is simultaneous delivery of a multiplicity of drugs with complimentary effects.

A further object of the present invention is to remove LDL from plasma by the LDL receptor pathway and deliver it to target tissues only.

Another object of the present invention is its application for diagnostic imaging of circulating bacteria and viruses.

Another object of the present invention is its application for therapeutic delivery of antibacterial and antiviral agents in a tissue specific manner at target sites.

These and other objects of the invention may become apparent to those skilled in the art upon review of the description of the invention as set forth hereinafter, in view of its drawing.

### BRIEF DESCRIPTION OF SEVERAL VIEWS OF THE DRAWINGS

FIG. 1a shows micelle carrying drug molecule in its core and a hydrophilic corona with LDL receptor, and a peptide that induces fusion or lysis of membrane vesicles.

FIG. 1b shows viruses attached to a triblock copolymer vesicle.

FIG. 1c shows a virus docking a triblock copolymer vesicle and passing DNA material into the vesicle.

FIG. 2 shows larger micelle aggregate carrying drug molecule in its core and a hydrophilic corona with LDL receptor with attached LDL molecules.

FIG. 3 shows circulating micelles aggregates with LDL molecules in the blood stream of a patient.

FIG. 4 shows endocytosis and subsequent endosomal permeation or destabilization for drug and LDL molecule delivery into the cell.

FIG. 5 shows the schematic diagram and flow chart of the present invention.

### DETAILED DESCRIPTION OF THE INVENTION

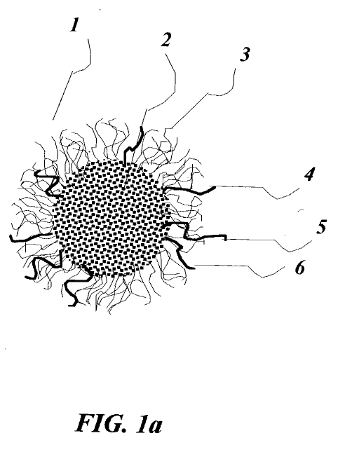

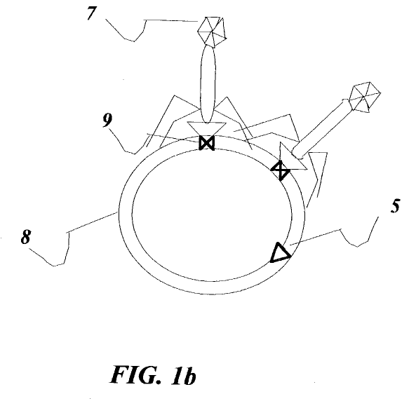

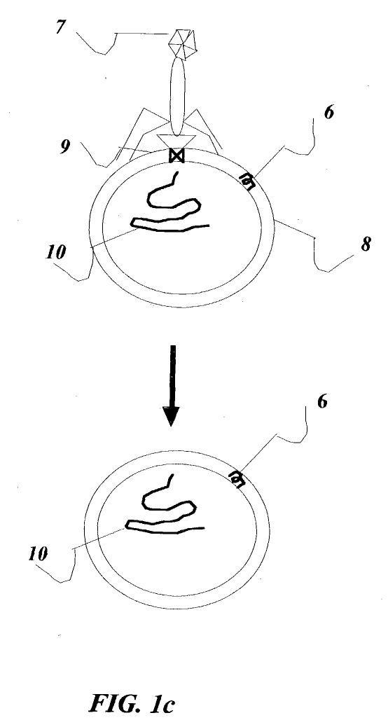

FIG. 1a shows micelle 1 carrying hydrophobic drug molecule in its core or in some desirable cases a placebo 2 and a hydrophilic corona 3 with LDL receptor 4, and a peptide that induces fusion or lysis 5 of membrane vesicles or membrane tranduction protein 6. The '221 patent described in detail the synthesis of a nanoparticle similar to the micellel used for the present invention. An example of an LDL receptor 4 has been described in detail by the '654 patent including the encoding nucleic acid. Peptides that induce fusion or lysis 5 of membrane vesicles has been described by Wagner E in Advanced Drug Delivery Review 1999, volume 38, page 279, or transduction domain peptides 6 as described by Jensen K D, Nori A, Tijerina M, Kopeckova P, Kopecek J, in Journal of Controlled Release, 2003, volume 87 page 89. The procedure is familiar to anyone skilled in the art. Each peptide could be made to be tissue specific such that tissues where LDL reduction is desirable have corresponding fusion and lysis receptors but are lacking for tissues where LDL cholesterol perform vital biochemical roles. Thereby improving tissue specificity of the cholesterol lowering drug.

FIG. 1b shows a virus 7 attached to a triblock copolymer vesicle 8 containing inserted channel protein 9 for effective docking as described by Graff A, Sauer M, Gelder P V, Meier W, in Proceedings of the National Academy of Sciences of the United States of America, 2002, volume 99, page 5064, and also inserted transduction domain peptides 6.

FIG. 1c shows a virus 7 docking via a channel protein 9 to a triblock copolymer vesicle 8 and passing DNA material 10 into the vesicle. The copolymer vesicles are transduced across the plasma membrane using transduction domain peptides 6.

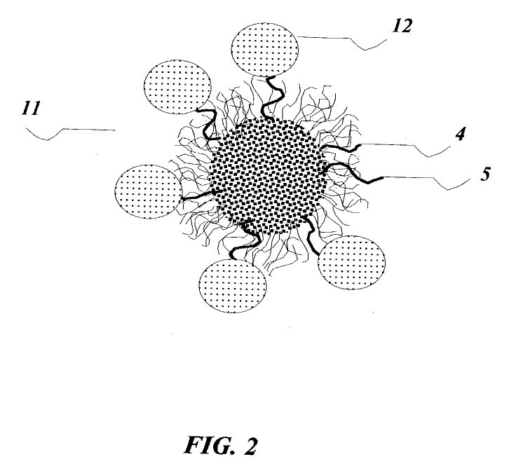

FIG. 2 shows large micelle aggregate 11 carrying drug molecule in its core 2 and a hydrophilic corona 3 with LDL receptor 4 with attached LDL molecules 12. There is a an attached peptide that induces fusion 5 or lysis 6 of membrane vesicles. The micelle aggregates 11 with LDL molecules 12 are echogenic because of high concentration of fatty molecules. They as well generate high intensity signals on magnetic resonance image

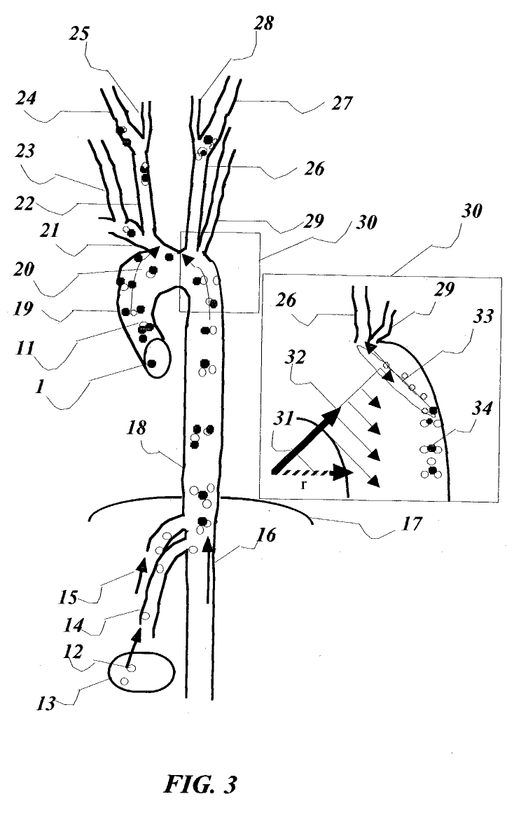

FIG. 3 shows circulating micelles with LDL receptors 1 introduced into blood and comes in contact with circulating LDL moleculesl2 forming micelle aggregates 11 with LDL molecules. The LDL molecules 12 could be formed in the endothelial cells 13 of the intestine and enter the blood stream through the branches of the mesentericl 14 and celiac 15 vessels after absorption. LDL molecules are also formed in the liver and circulate via the portal system. Some L DL molecules because of their high floatation constants Sƒ might rise in blood column of the abdominal aorta 16 above the diaphragm 17 to the descending aorta 18 coming in contact with the micellel in blood forming micelle aggregates. The micelle aggregates 11 with LDL molecules will rise through the aortic arch 19 slower than the blood flow jet always at the outer curvature 20 and would enter the brachiocephalic artery 21 an then into the right common carotid artery (RCCA) 22 and the right vertebral artery (RVA) 23. As the micelle aggregates rise along the RCCA 22 to the carotid bulb and into the right internal carotid artery (RICA) 24 and right external carotid artery (RECA) 25. In addition, quantitative detection of the micelle aggregates with LDL molecules could be performed using microembolic signal detection algorithm of transcranial Doppler ultrasonography using commercially available instruments such as Multi-Dop T (EME Sipplingen, Germany). Imaging of the micelle aggregates with LDL molecules in the RCCA 22, RVA 23, RICA 24, RECA 25 and could be performed using color flow Doppler ultrasound and B-mode using available commercial systems such as Genesis C FM (Biosound, Indianapolis, Ind.), and magnetic resonance imaging Magnetom (Siemens, Erlangen, Germany). Similarly imaging micelle aggregates could be performed in the left common carotid (LCCA) 26, left internal carotid artery (LICA) 27, left external carotid artery (LECA) 28 and left vertebral artery (LVA) 29. Flow at aortic arch 30 will show peculiarities for flow at curvatures 31 with radius r and primary flow profile 32 will develop along with secondary flow profile and helical patterns 33 involving retrograde rising micelle aggregates 34 into a helical flow vortex 33 which eventually propels the micelles into the LCCA 26, LVA 29, LICA 27 and LECA 28.

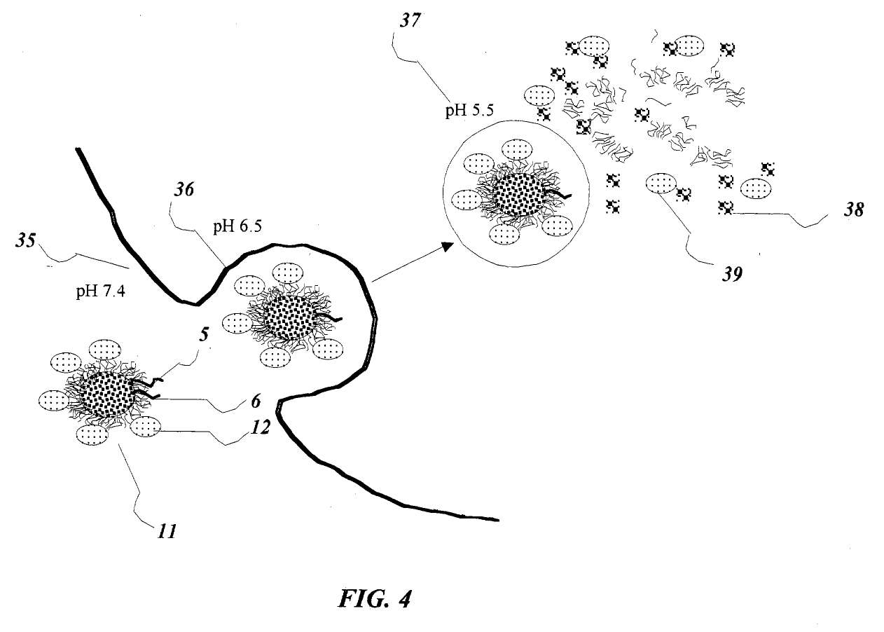

FIG. 4 shows endocytosis and subsequent endosomal permeation or destabilization for drug and LDL molecule delivery into the cell. The micelle aggregates 11 with LDL molecules 12 move from the extracellular space 35 with pH of 7.4 by way of example. It fuses with the cell in a receptor mediated process using fusion protein 5. The micelle aggregates enters into the cell 36 by the process of endocytosis and subsequent endosomal permeation 37 and at lower pH 5.5 destabilize delivering the drug 38 and LDL molecules 39. Drugs such as statins inhibit HMG-CoA reductase and prevent the biosynthesis of cholesterol by way of example. In tissues where LDL-cholesterol is desirable fusion does not occur. In some cases, it may be desirable to enhance cholesterol uptake by specific tissues such as the adrenals and in such cases tissue specific fusion proteins 5 could facilitate entry of the micelle aggregates but lacking the drug substances in its core into the cells. Similarly, desirable levels of cholesterol could be maintained in muscle tissue and prevent serious side effects of statins.

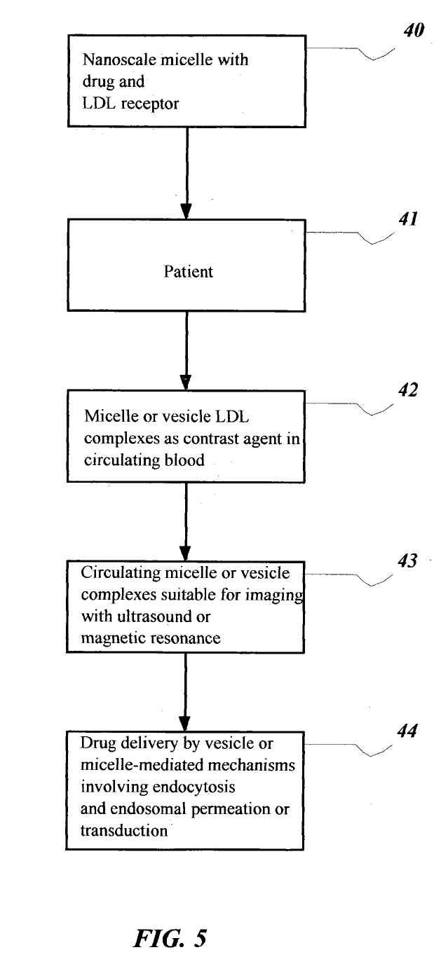

FIG. 5 shows the schematic diagram and flow chart of the present invention. Nanoscale micelle with drug and LDL receptor40 is introduced into blood of patient 41 and forms micelle-LDL complexes which act as a contrast agent in circulating blood 42 and could be demonstrated using ultrasound or magnetic resonance imaging 43. The drug within the micelle is delivered by micelle mediated mechanisms involving endocytosis and endosomal permeation 44 using specifically designed fusionS or lysis proteins 6 for tissue targeting.

While the preferred embodiment of the present invention is described above, it is contemplated that numerous modification may be made thereto for particular applications without departing from the spirit and scope of the present invention. Accordingly, it is intended that the embodiment described be considered only as illustrative of the present invention and that the scope thereof should not be limited thereto but be determined by reference to the claims hereinafter provided.

1. A method and system for improving diagnostic imaging and/or delivering therapeutically active agents used in control of hyperlipidemia, comprising:
(a) assembling a nanoscale container having a partition of hydrophobic active ingredient of a drug in the core of a micelle;
(b) simultaneously with (a) and a hydrophilic corona surrounding the core yielding spherical micelles;
(c) incorporating LDL receptors into the corona;
(d) incorporating tissue specific peptides that induce fusion or lysis of membrane;
(e) introducing the said micelles into the blood stream of a patient with circulating LDL molecules in the blood stream;
(f) forming micelle aggregates with circulating LDL molecule
(g) determining the presence of the micelle aggregates by interaction with ultrasound irradiation or magnetic resonance to generate a detectable signal
(h) entering the targeted cells by endocytosis and delivering the therapeutically active agent and LDL molecules into the cytoplasm;
(i) simultaneously with (h) lowering the concentration circulating LDL molecules:
(j) simultaneously with (i) lowering the LDL-cholesterol synthesis only in specific tissues.

2. The invention of claim 1, wherein the therapeutically active agent is a drug included in the core of the micelle, that is formulated for immediate release, pulsatile release, controlled release, extended release, delayed release, targeted release, or targeted delayed release

3. The invention of claim 1, wherein the therapeutically active agent is delivered to the general circulation to prevent excessive extraction by the liver and improving the bioavailability.

4. The invention of claim 1, wherein the copolymer micelle aggregates are echogenic due to high lipid content on ultrasound images and generate hyper intense signals on magnetic resonance images.

5. The invention of claim 1, wherein the drug incorporated in the core of the micelle comprise a statin.

6. The invention of claim 1, wherein the tissue specific fusion or lysis protein ensures tissue differential elimination of cholesterol.

7. The invention of claim 1, wherein there is quantitative and qualitative determination of LDL molecules within the blood stream using imaging methods.

8. The invention of claim 1, wherein LDL is removed from plasma by the LDL receptor pathway and delivered to target tissues only.

9. A method and system for improving diagnostic imaging and/or delivering therapeutically active agents used in control of infectious diseases, comprising:
(a) assembling a nanoscale container having a partition of hydrophobic active ingredient of a drug in the core of a micelle;
(b) simultaneously with (a) and a hydrophilic corona surrounding the core yielding spherical micelles;
(c) incorporating into the corona receptors for lipopolysacchride bacterial coat or glycoprotein for viral envelope;
(d) incorporating tissue specific peptides that induce fusion or lysis of membranes;
(e) introducing the said micelles into the blood stream of a patient infected with a pathologic microorganism;
(f) forming micelle aggregates with circulating microorganism
(g) determining the presence of the micelle aggregates by interaction with ultrasound irradiation or magnetic resonance to generate a detectable signal
(h) entering the targeted tissue cells that are lesion sites by endocytosis or transduction and delivering the therapeutically active agents into the cytoplasm;
(i) simultaneously with (h) eliminating the circulating microorganisms from the bloodstream
(j) simultaneously with (i) concentrating the therapeutically active agents only at lesion sites in specific tissues.

10. The invention of claim 9, wherein the suitable hydrophobic active ingredients are selected from a group consisting of analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta-Blockers, cardiac inotropic agents, corticosteroids, diuretics, anti-parkinsonian agents, gastrointestinal agents, histamine receptor antagonists, keratolytics, lipid regulating agents, anti-anginal agents, cox-2 inhibitors, leukotriene inhibitors, macrolides, muscle relaxants, nutritional agents, opioid analgesics protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

11. The invention of claim 9, wherein specific, non-limiting examples of suitable hydrophobic active ingredients may be selected from: acetretin, albendazole, albuterol, aminoglutethimide amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacit cisapride, clarithromycin, clemastine, clomiphene, clomipramine, clopidogrel, codeine coenzyme Q10, cyclobenzapmme, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, furazolidone, gabapentin, gemfibrozil, glibenclamide, glipizide, glyburide, glimepiride griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukasi nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamir, E, vitamin K, zafirlukast, zileuton, zolmitriptan, zolpidem, zopiclone, salts, isomers and derivatives of the above-listed hydrophobic active ingredients may also be used, as well as mixtures.

12. The invention of claim 9, wherein the hydrophilic active ingredient can be a cytokine, a peptidomimetic, a peptide, a protein, a toxoid, a serum, an antibody, a vaccine, a nucleoside, a nucleotide, a portion of genetic material, a nucleic acid, or a mixture thereof

13. The invention of claim 9, wherein, non-limiting examples of suitable hydrophilic active ingredients that could potentially be enclosed in the corona of the micelle are selected from: acarbose, acyclovir, acetyl cysteine, acetylcholine chloride, alatrofloxacin, alendronate, aglucerase, amantadine hydrochloride, ambenomium, amifostine, amiloride hydrochloride, aminocaproic acid, amphotericin B, antihemophilic factor (human), antihemophilic factor (porcine), antihemophilic factor (recombinant), aprotinin, asparaginase, atenolol, atracurium besylate, atropine, azithromycin, aztreonam, BCG vaccine, bacitracin, becalermin, belladona, bepridil hydrochloride, bleomnycin sulfate, calcitonin human, calcitonin salmon, carboplatin, capecitabine, capreomycin sulfate, cefamandole nafate, cefazolin sodium, cefepime hydrochloride, cefixime, cefonicid sodium, cefoperazone, cefotetan disodium, cefotaxime, cefoxitin sodium, ceftizoxime, ceftriaxone, cefuroxime axetil, cephalexin, cephapirin sodiurr cholera vaccine, chorionic gonadotropin, cidofovir, cisplatin, cladribine, clidinium bromide clindamycin and clindamycin derivatives, ciprofloxacin, clodronate, colistimethate sodium, colistin sulfate, corticotropin, cosyntropin, cromolyn sodium, cytarabine, dalteparin sodium, danaparoid, desferrioxamine, denileukin diflitox, desmopressin, diatrizoate meglumine and diatrizoate sodium, dicyclomine, didanosine, dirithromycin, dopamine hydrochloride, dornase alpha, doxacurium chloride, doxorubicin, etidronate disodium, enalaprilat, enkephalin, enoxaparin, enoxaprin sodium, ephedrine, epinephrine, epoetin alpha, erythromycin, esmolol hydrochloride, factor IX, famciclovir, fludarabine, fluoxetine, foscarnet sodium, ganciclovir granulocyte colony stimulating factor, granulocyte-macrophage stimulating factor, growth hormones—recombinant human, growth hormone—bovine, gentamycin, glucagon, glycopyrolate, gonadotropin releasing hormone GnRH and synthetic analogs thereof, gonadorelin, grepafloxacin, hemophilus B conjugate vaccine, Hepatitis A virus vaccine inactivated, Hepatitis B virus vaccine inactivated, heparin sodium, indinavir sulfate, influenza virus vaccine, interleukin-2, interleukin-3, insulin-human, insulin lispro, insulin procine, insulin NPH, insulin aspart, insulin glargine, insulin detemir, interferon alpha, interferon beta, ipratropium bromide, ifosfamide, Japanese encephalitis virus vaccine, lamivudine, leucovorin calcium, leuprolide acetate, levofloxacin, lincomycin and lincomycin derivatives, lobucavir lomefloxacin, loracarbef, mannitol, measles virus vaccine, meningococcal vaccine, menotropins, mepenzolate bromide, mesalamine, methenamine, methotrexate, methscopolamine, metformin hydrochloride, metoprolol, mezocillin sodium, mivacurium chloride, murnps viral vaccine, nedocromil sodium, neostigmine bromide, neostigmine methy sulfate, neurontin, norfloxacin, octreotide acetate, ofloxacin, olpadronate, oxytocin, pamidronate disodium, pancuronium bromide, paroxetine, perfloxacin, pentamidine isethionate, pentostatin, pentoxifylline, periciclovir, pentagastrin, pentholamine mesylate, phenylalanine, physostigmine salicylate, plague vaccine, piperacillin sodium, platelet derived growth factor-human, pneumococcal vaccine polyvalent, poliovirus vaccine inactivated, poliovirus vaccine live (OPV), polymyxin B sulfate, pralidoxime chloride, pramlintide, pregabalin, propafenone, propenthaline brormide, pyridostigmine bromide, rabies vaccine, residronate, ribavarin, rimantadine hydrochloride, rotavirus vaccine, salmeterol xinafoate, sinealide, small pox vaccine, sotalol, somatostatin, sparfloxacin, spectinomycin, stavudine, streptokinase, streptozocin, suxamethonium chloride, tacrine hydrochloride, terbutaline sulfate, thiopeta, ticarcillin, titudronate, timolol, tissue type plasminogen activator, TNFR:Fc, TNK-TPA, trandolapril, trimetrexate gluconate, trospectinomycin, trovafloxacin, tubocurarine chloride, tumor necrosis factor, typhoid vaccine live, urea, urokinase, vancomycin, valacyclovir, valsartan, varicella virus vaccine live, vasopressin and vasopressin derivatives, vecuronium bromide, vinblastine, vincristine, vinorelbine, vitamin B12, warfarin sodium, yellow fever vaccine, zalcitabine, zanamivir, zolendronate, zidovudine, pharmaceutically acceptable salts, isomers and derivatives thereof, and mixtures thereof.

14. A method and system for improving diagnostic imaging and/or delivering therapeutically active agents used in control of infectious diseases, comprising:
(a) assembling nanoscale diblock copolymer vesicles having a hydrophobic and a hydrophilic segment;
(b) inserting a natural channel protein in its membrane for docking of viruses or bacteria
(c) inserting tissue specific membrane proteins for lysis or fusion;
(d) introducing the said copolymer vesicles into the blood stream of a patient with circulating pathologic microorganisms;
(e) forming vesicle aggregates with circulating pathologic microorganism
(f) determining the presence of the vesicle aggregates with microorganisms in the vessels using an imaging method;
(g) entering the targeted cells by endocytosis or transduction and delivering the therapeutically active agents into the cytoplasm of targeted tissue sites;
(h) simultaneously with (g) eliminating the microorganism in the bloodstream
(i) simultaneously with (h) raising the concentration of the therapeutically active agents only in specific tissues at lesion sites to prevent drug resistance and toxicity.

15. The invention of claim 14, wherein block copolymer vesiclescarrying an aqueous drug and delivering high concentrations of the drug to the bloodstream for longer circulation time and improving the bioavailability of the drug.

16. The invention of claim 14, wherein the therapeutically active agents are deoxyribonucleic acid using virus assisted loading into copolymer vesicles and subsequently using tissue specific fusion or lysis protein to reach cells of target tissues.

17. The invention of claim 14, wherein, non-limiting examples of suitable hydrophobic active ingredients are selected from: acetretin, albendazole, albuterol, aminoglutethimide, amiodarone amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine chlorpheniramine, cholecalciferol, cilostazol, cimetidine, cinnarizine, ciprofloxacin, cisapride clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, furazolidone, gabapentin, gemfibrozil, glibenclamide, glipizide, glyburide, glimepiride, griseofulvin, halofantrin, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide loratadine, lovastatin, L-thryroxine, lutein, lycopene, medroxyprogesterone, mifepristone, mefloquine, megestrol acetate, methadone, methoxsalen, metronidazole, miconazole midazolam, miglitol, minoxidil, mitoxantrone, montelukast, nabumetone, nalbuphine naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxac in, ubidecarenone, valsartan, venlafaxine, verteporfin, vigabatrin, vitamin A, vitamin D, vitamin E, vitamin K, zafirlukast, zileuton, zolmitriptan, zolpidem, zopiclone, pharmaceutically acceptable salts, isomers and derivatives thereof, and mixtures thereof.

17. The system of claim 14, wherein the hydrophilic active ingredient can be a cytokine, a peptidomimetic, a peptide, a protein, a toxoid, a serum, an antibody, a vaccine, a nucleoside, a nucleotide, a portion of genetic material, a nucleic acid, or a mixture thereof.

18. The invention of claim 14, wherein the hydrophilic active ingredients are selected from the group consisting acarbose, acyclovir, acetyl cysteine, acetylcholine chloride, alatrofloxacin, alendronate, aglucerase, amantadine hydrochloride, ambenomium; amifostine, amiloride hydrochloride, aminocaproic acid, amphotericin B, antihemophilic factor (human), antihemophilic factor (porcine), antihemophilic factor (recombinant), aprotinin, asparaginase, atenolol, atracurium besylate, atropine, azithromycin, aztreonam, BCG vaccine, bacitracin, becalermin, belladona, bepridil hydrochloride, bleomnycin sulfate, calcitonin human, calcitonin salmon, carboplatin, capecitabine, capreomycin sulfate, cefamandole nafate, cefazolin sodium, cefepime hydrochloride, cefixime, cefonicid sodium, cefoperazone, cefotetan disodium, cefotaxime, cefoxitin sodium, ceftizoxime, ceftriaxone, cefuroxime axetil cephalexin, cephapirin sodium, cholera vaccine, chorionic gonadotropin, cidofovir, cisplatin, cladribine, clidinium bromide, clindamycin and clindamycin derivatives, ciprofloxacin, clodronate, colistimethate sodium, colistin sulfate, corticotropin, cosyntropin, cromolyn sodium, cytarabine, dalteparin sodium, danaparoid, desferrioxamine, denileukin diflitox, desmopressin, diatrizoate meglumine and diatrizoate sodium, dicyclomine, didanosine, dirithromycin, dopamine hydrochloride, dornase alpha, doxacurium chloride, doxorubicin, etidronate disodium, enalaprilat, enkephalin, enoxaparin, enoxaprin sodium, ephedrine, epinephrine, epoetin alpha, erythromycin, esmolol hydrochloride, factor IX, famciclovir, fludarabine, fluoxetine, foscamet sodium, ganciclovir, granulocyte colony stimulating factor, granulocyte-macrophage stimulating factor, growth hormones—recombinant human, growth hormone—bovine, gentamycin, glucagon, glycopyrolate, gonadotropin releasing hormone GnRH and synthetic analogs thereof, gonadorelin, grepafloxacin, hemophilus B conjugate vaccine, Hepatitis A virus vaccine inactivated, Hepatitis B virus vaccine inactivated, heparin sodium, indinavir sulfate, influenza virus vaccine, interleukin-2, interleukin-3, insulin-human, insulin lispro, insulin procine, insulin NPH, insulin aspart, insulin glargine, insulin detemir, interferon alpha, interferon beta, ipratropium bromide, ifosfamide, Japanese encephalitis virus vaccine, lamivudine, leucovomm calcium, leuprolide acetate, levofloxacin, lincomycin and lincomycin derivatives, lobucavir, lomefloxacin, loracarbef, mannitol, measles virus vaccine meningococcal vaccine, menotropins, mepenzolate bromide, mesalamine, methenamine, methotrexate, methscopolamine, metformin hydrochloride, metoprolol, mezocillin sodium, mivacurium chloride, mumps viral vaccine, nedocromil sodium, neostigmine bromide, neostigmine methyl sulfate, neurontin, norfloxacin, octreotide acetate, ofloxacin, olpadronate, oxytocin, pamidronate disodium, pancuronium bromide, paroxetine, perfloxacin, pentamidine isethionate, pentostatin, pentoxifylline, periciclovir, pentagastrin, pentholamine mesylate, phenylalanine, physostigmine salicylate, plague vaccine, piperacillin sodium, platelet derived growth factor-human, pneumococcal vaccine polyvalent, poliovirus vaccine inactivated, poliovirus vaccine live (OPV), polymyxin B sulfate, pralidoxime chloride, pramlintide, pregabalin, propafenone, propenthaline bromide, pyridostigmine bromide, rabies vaccine, residronate, ribavarin, rimantadine hydrochloride, rotavirus vaccine, salmeterol xinafoate, sinealide, small pox vaccine, sotalol, somatostatin, sparfloxacin, spectinomycin, stavudine, streptokinase, streptozocin, suxamethonium chloride, tacrine hydrochloride, terbutaline sulfate, thiopeta, ticarcillin, tiludronate, timolol, tissue type plasminogen activator, TNFR:Fc, TNK-tPA, trandolapril, trimetrexate gluconate, trospectinomycin, trovafloxacin, tubocurarine chloride, tumor necrosis factor, typhoid vaccine live, urea, urokinase, vancomycin, valacyclovir, valsartan, varicella virus vaccine live, vasopressin and vasopressin derivatives, vecuronium bromide, vinblastine, vincristine, vinorelbine, vitamin B12, warfarin sodium, yellow fever vaccine, zalcitabine, zanamivir, zolendronate, zidovudine, pharmaceutically acceptable salts, isomers and derivatives thereof, and mixtures thereof.

19. The invention of claim 14, wherein the composition of the drug included as active ingredient is formulated for immediate release, pulsatile release, controlled release, extended release, delayed release, targeted release, or targeted delayed release.

20. The invention of claim 14, wherein there is therapeutic delivery of antibacterial and antiviral agents in a tissue specific manner at target lesion sites.
